(19) United States
(12) Patent Application Publication
(10) Pub. No.: US 2003/0112981 A1
(43) Pub. Date: Jun. 19, 2003

(54) Active noise control with on-line-filtered C modeling

(75) Inventors: Richard D. McWilliam, Shedden (CA); Ian McLean, Chatham (CA)

Correspondence Address: SIEMENS CORPORATION, INTELLECTUAL PROPERTY LAW DEPARTMENT, 170 WOOD AVENUE SOUTH, ISELIN, NJ, 08830, US

(73) Assignee: Siemens VDO Automotive, Inc.

(21) Appl. No.: 10/309,829

(22) Filed: Dec. 4, 2002

Related U.S. Application Data
(60) Provisional application No. 60/341,532, filed on Dec. 17, 2001.

(51) Int. Cl. A61F 11/06; G10K 11/16; H03B 29/00

(52) U.S. Cl. 381/71.11

(57) ABSTRACT

A method of noise attenuation for a vehicle comprises generating a noise attenuating sound based on an assumption through speaker 18. (See FIG. 1). A test signal is generated comprising a frequency range of sounds desired to be attenuated for obtaining actual data. The test signal is received by microphone 26 and then filtered by filter 30. The assumption is then assessed based upon the filtered received test signal. The noise attenuating sound 32 is altered based on the assessment.

This application claims priority to Provisional Patent Application Serial No. 60/341,532 filed on Dec. 17, 2001.

### BACKGROUND OF THE INVENTION

This invention relates to an active method and system for controlling automotive induction noise.

Manufacturers have employed active and passive methods to reduce engine noise within the passenger compartment. Such noise frequently emanates from the engine, travels through the air induction system and emanates out of the mouth of the air intake into the passenger compartment. Efforts have been made to reduce the amount of engine noise traveling through the air induction system. These efforts include the use of both passive devices such as expansion chambers and Helmholtz resonators and active devices involving anti-noise generators.

Active systems use a speaker to create a canceling noise that attenuates engine noise. The sound created is out of phase with the engine noise and combines with this noise to result in its reduction. Generally, this sound is generated in proximity to the mouth of the air induction system.

An active noise control system using feed forward control comprises a speaker, an error microphone, a reference sensor and a control unit. Prior to active noise control operation, the control unit measures a digital model of an electrical/acoustic signal path, known as a C-model, of the various components of the system. This path includes all of the elements that an electric/acoustic signal will pass from the digital/analog output of the control unit, the electrical path to the audio amplifier, the audio amplifier, the electrical path to the speaker, the speaker, the acoustic transmission path from the speaker to the error microphone, the error microphone, and the electrical path from the error microphone to the analog/digital input of the control unit.

The control unit measures the C-model by sending a known broadband signal (a “test signal”) from the digital/analog output of the control unit (an “input signal”) through the electrical/acoustic path back to the analog/digital input of the control unit (an “output signal”). The control unit samples the transmitted signal at the analog/digital input. A frequency response function is then calculated as known from the measured ratio of the sampled output signal to the known input signal.

The control unit then computes a digital filter model with the same frequency response function as that measured for the control path, the C-model. This digital filter model is then used in a software algorithm to create the active noise attenuating signal of the system. As known, the software algorithm further obtains input from the error microphone and the reference signal regularly during operation of the noise attenuation system so that a control signal may be sent to the speaker to attenuate noise on a continuous basis.

The accuracy of the C-model is influenced by a number of factors. For example, a change in environmental conditions, such as air temperature or atmospheric pressure, will change the acoustic path from the speaker to the microphone, since this path is dependent upon the speed of sound. In addition, the responsiveness of the electrical/mechanical components will also change not only as a consequence of changing environmental conditions but also such factors as component aging. As a result, the C-model must be continuously updated. This updating is accomplished by adding the test signal (the broadband modeling noise) to the noise attenuating signal during active noise control operation. Because the error microphone senses only a small residual signal from the noise cancellation, most of the remaining signal that the error microphone senses comprises the test signal. The received test signal contain real time data concerning the system that permits the C-model to be updated.

In addition to picking up the test signal and the residual noise attenuating signal, the error microphone will also pick up background noise during on-line C-modeling. Consequently, the signal to noise ratio of the error microphone must be large enough to accurately measure the C-model. That is, when the vehicle is running, the test signal must be large enough so that it can be accurately measured against the engine noise also sensed by the error microphone. However, if the test signal is too large, it will be audible when transmitted from speaker to error microphone and possibly annoying to vehicle occupants. Thus, the level of the test signal must be kept low enough not to be noticeable yet large enough to permit acceptable signal to noise levels for an accurate on-line C-model.

One proposed solution to this problem is to generate the test signal only when the throttle is nearly closed so that background engine noise is minimized. Moreover, most of the engine noise is generated at discrete frequencies that are harmonics of the engine noise so that the signal to noise degeneration resulting from inaccuracies of the C-model only happens at these discrete frequencies. Thus, between these frequencies, C-modeling will be accurate. So, during on-line C-modeling, a small change in engine speed will reduce the C-model errors at frequencies associated with engine harmonics. However, these conditions do not result in very robust on-line C-modeling.

A need therefore exists for an improved noise attenuating method and system that permits accurate digital modeling during vehicle operation.

### SUMMARY OF THE INVENTION

The present invention comprises a system and method of noise attenuation. Like existing noise attenuation systems, the inventive system comprises a speaker and a control unit that permits the speaker to create a noise attenuating sound based on a digital model of the transmission of signals through the system. An error microphone picks up sound that is not attenuated. A test signal is generated to update the digital model with real time data. In contrast to existing systems, however, after the test signal is received by the error microphone, background noise is filtered from the test signal, permitting lower volume test signals to be used with the system.

The system may include a data input for the filter, which adjusts the filtering based on information received from the data input. For example, the data input may comprise a signal received by a sensor, such as a tachometer, which senses engine speed. In this way, the filter may be adjusted based on the anticipated level of engine sound to filter out this sound from the test signal. The filter may be a hardware or software filter. A digital signal processor may be used as a hardware filter.

With this system, a test signal is generated to assess real-time conditions of the system. When the test signal is received, it is filtered. The real-time conditions of the system are assessed from the filtered test signal. Further noise attenuating sound is then altered based upon this assessment of the filtered test signal.

The received test sound may be filtered for background noise, such as engine noise. The filter may be adjusted based on the speed of the engine. In addition, the test signal may comprise a frequency range of sounds to be attenuated. The frequency range may comprise a random selection of sounds, such as white noise.

Accordingly, the inventive system and method permits the removal of background noise from the test sound. Thus, the test signal may be lowered in volume to avoid the incursion of sound into the passenger compartment. Without significant additional cost, the method and system provides an improved technique for noise attenuation of a vehicle.

### BRIEF DESCRIPTION OF THE DRAWINGS

The various features and advantages of this invention will become apparent to those skilled in the art from the following detailed description of the currently preferred embodiment. The drawings that accompany the detailed description can be briefly described as follows:

FIG. 1 illustrates the inventive noise attenuation system.

FIG. 2 illustrates the method of noise attenuation for the system of FIG. 1.

### DETAILED DESCRIPTION OF THE PREFERRED EMBODIMENT

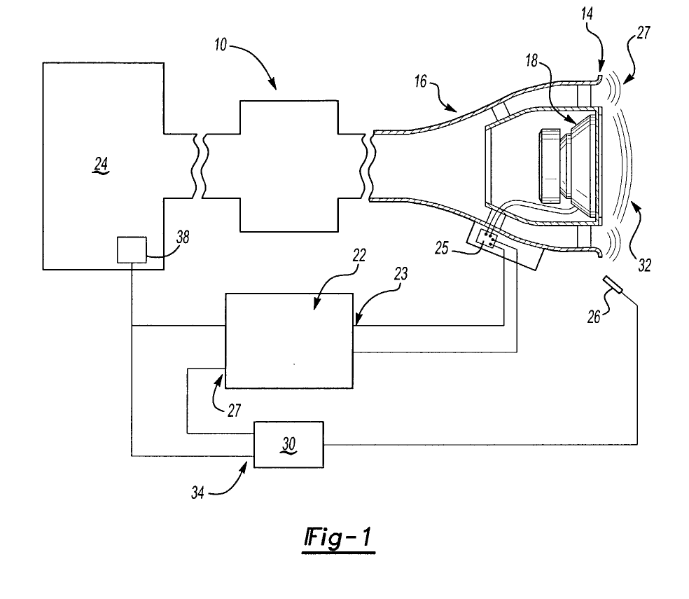

FIG. 1 illustrates inventive noise attenuation system 10 for a vehicle, such as an automobile. As known, air intake 14 permits the entry of air into air induction body 16, which passes air to engine 24. Engine noise 27 may emanate from the mouth of air intake 14 and ultimately to a passenger compartment. To reduce this level of noise, speaker 18 is controlled by control unit 22 to create noise attenuating sound 32, which comprises a sound wave out of phase with engine noise 27.

However, noise attenuating sound 32 may be insufficient to attenuate engine noise 27. Accordingly, microphone 26, an error microphone, picks up sound that is not attenuated. Error microphone 26 is in communication with control unit 22, which adjusts noise attenuating sound 32 based on the signal received by a microphone 26. These features of noise attenuation system 10 are known.

Also known in the art, during operation, control unit 22 measures a digital model of an electrical/acoustic signal path, known as a C-model, of the various components of the system. This path includes all of the elements that an electric/acoustic signal will pass from the digital/analog output 23 of control unit 22, the electrical path to audio amplifier 25, audio amplifier 25, the electrical path to speaker 18, speaker 18, the acoustic transmission path from speaker 18 to error microphone 26, error microphone 26, and the electrical path from error microphone 26 to analog/digital input 27 of control unit 22.

Control unit 22 measures the C-model by sending a known broadband signal (a “test signal”) from digital/analog output 23 of control unit 22 (an “input signal”) through the electrical/acoustic path back to analog/digital input 27 of control unit 22 (an “output signal”). Control unit 22 samples the transmitted test signal at analog/digital input 27. A frequency response function is then calculated as known from the measured ratio of the sampled output signal to the known input signal.

Control unit 22 then computes a digital filter model with the same frequency response function as that measured for the control path, the C-model. This digital filter model is then used in a software algorithm to create the active noise attenuating signal of the system. As known, the software algorithm further obtains input from microphone 26 and a reference signal from engine speed sensor 38 regularly during operation of the noise attenuation system so that a control signal may be sent to the speaker to attenuate noise on a continuous basis.

As known, a test signal is sent periodically to obtain updated data or real-time data of existing system conditions, such as conditions that may change due environmental conditions, aging of components, and other factors. This test signal may be sent by adding the test signal to the noise attenuating signal during active noise control operation. Because a test signal travels through system 10, including its electrical/mechanical components and their environment, the test signal is affected by the real time conditions of system 10 caused by the physical environment and its effect on the electrical/mechanical components, the age of the electrical/mechanical components, and other changing system conditions. Such real time data may then be used to update and recalibrate control unit 22 based on this real time data, thereby altering noise canceling sound 32 to account for the changed system conditions.

However, when the test signal is generated during vehicle operation, engine noise 27 from engine 24 may interfere with the reception of test signal by microphone 26. In contrast to existing noise attenuation systems, noise attenuation system 10 further employs filter 30 to filter out background noise, such as engine noise 27, from test signal. In this way, the test signal may be received by control unit 22 without background noise, thereby permitting a lower volume test signal to be used. Moreover, control unit 22 may inject test signal at any time rather than when throttle is nearly closed because filter 30 filters out engine noise 27, which ordinarily may interfere with reception of test signal.

Filter 30 may comprise software or preferably a hardware filter. Filter 30 is in communication with microphone 26 and picks up test signal from speaker 18. Filter 30 then filters out background noise through known filters such as the Kalman filter, Vold-Kalman, order tracking filtering or any equivalent and known filter. Filter 30 removes harmonic engine noises or other noise sources from the signal received by microphone 26. Filter 30 then communicates the filtered signal to control unit 22.

In addition, filter 30 may have data input 34, which receives information from engine speed sensor 38, here a tachometer, which provides information to filter 30, such as engine speed, to permit the altering of filtering based on this information. The resulting filter 30 thus greatly eliminates engine noise and background sound from test signal.

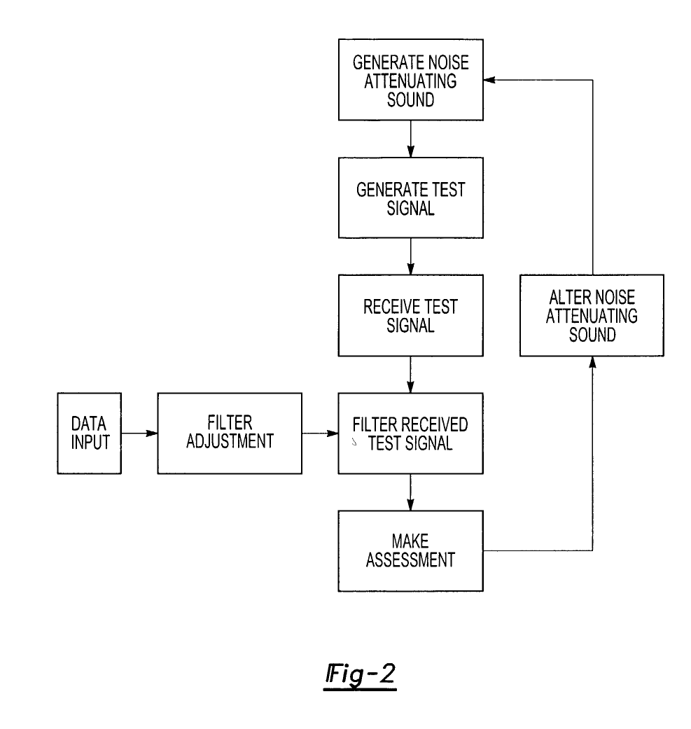

FIG. 2 illustrates the inventive technique. As known, a noise attenuating sound is generated. To improve attenuation, a test signal is generated as well. Preferably, the test signal comprises random sounds selected from a frequency range of sounds to be attenuated. For example, the sounds may comprise the different frequencies of sounds that may emanate from engine 24. These random sounds may create white noise and result in an improved test signal for analysis.

The test signal is received and then filtered of background noise, such as engine noise. The filter maybe adjusted based on data input from a source such as sensor 38, a tachometer. Once the background noise is filtered out, an assessment of the test signal is made and the noise attenuating sound altered based on real-time conditions. The resulting techniques permits a C-model to more accurately represent existing system conditions and thereby improve noise attenuation.

The aforementioned description is exemplary rather that limiting. Many modifications and variations of the present invention are possible in light of the above teachings. The preferred embodiments of this invention have been disclosed. However, one of ordinary skill in the art would recognize that certain modifications would come within the scope of this invention. Hence, within the scope of the appended claims, the invention may be practiced otherwise than as specifically described. For this reason the following claims should be studied to determine the true scope and content of this invention.

What is claimed is:

1. A method of noise attenuation for a vehicle engine, comprising the steps of:
generating a noise attenuating sound based on an assumption;
generating a test signal comprising a frequency range of sounds to be attenuated;
receiving the test signal;
filtering the received test signal;
assessing the assumption based on the filtered received test signal; and
altering the noise attenuating sound based on the assessment wherein filtering the received test signal comprises filtering a background noise from the test signal.

2. The method of noise attenuation of claim 1 wherein at least a portion of the background noise comprises engine noise from an engine.

3. The method of noise attenuation of claim 2 including the step of adjusting the filtering based on a speed of the engine.

4. The method of noise attenuation of claim 1 wherein the frequency range of sounds comprises a random selection of sounds.

5. The method of noise attenuation of claim 4 wherein the random selection of sounds comprises white noise.

6. A method of noise attenuation, comprising the steps of:
generating a noise attenuating sound based on an assumption;
generating a test signal;
receiving the test signal;
filtering the received test signal;
assessing the assumption based on the filtered received test signal; and
altering the noise attenuating sound based on the assessment.

7. The method of noise attenuation of claim 6 wherein filtering the received test signal comprises filtering a background noise from the test signal.

8. The method of noise attenuation of claim 7 wherein at least a portion of the background noise comprises engine noise from an engine.

9. The method of noise attenuation of claim 8 including the step of adjusting the filtering based on a speed of the engine.

10. The method of noise attenuation of claim 6 wherein the test signal comprises a frequency range of sounds desired to be attenuated.

11. The method of noise attenuation of claim 10 wherein the frequency range of sounds comprises a random selection of sounds.

12. The method of noise attenuation of claim 11 wherein the random selection of sounds comprises white noise.

13. A noise attenuation system for a vehicle, comprising:
a speaker;
a control unit controlling said speaker to create a noise attenuating sound;
a microphone in communication with said control unit; and
a filter in communication with said microphone and said control unit for filtering sounds received by said microphone.

14. The noise attenuation system of claim 13 including a data input to said filter.

15. The noise attenuation system of claim 14 wherein said filter adjusts based on said data input.

16. The noise attenuation system of claim 15 wherein said data input comprises a signal received by a sensor.

17. The noise attenuation system of claim 16 wherein said sensor comprises a tachometer.

18. The noise attenuation system of claim 13 wherein said filter comprises a software filter.

19. The noise attenuation system of claim 13 wherein said filter comprises a hardware filter.

20. The noise attenuation system of claim 19 wherein said hardware filter comprises a digital signal processor.
